(19) United States
(12) Patent Application Publication
(10) Pub. No.: US 2008/0096786 A1
(43) Pub. Date: Apr. 24, 2008

(54) Shampoo Compositions Containing Cationic Polymer and an Anionic Surfactant Mixture

(76) Inventors: Neil Christopher Holt, Wirral (GB); Neil Scott Shaw, Wirral (GB)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 11/791,782

(22) PCT Filed: Nov. 25, 2005
(86) PCT No.: PCT/EP05/12862

§ 371 Date: May 29, 2007

(30) Foreign Application Priority Data
Dec. 4, 2004 (EP) 04257558.9

(51) Int. Cl. A61K 8/46; A61K 8/73; A61K 8/81; A61Q 5/02; A61Q 5/12; C11D 1/12; C11D 1/37

(52) U.S. Cl. 510/275

(57) ABSTRACT

The invention provides an aqueous shampoo composition comprising, in admixture: (i) a first anionic surfactant in which the ratio between the molecular weights of the polar (hydrophilic) and non-polar (hydrophobic) parts of the molecule is less than 1.5; (ii) a second anionic surfactant in which the ratio between the molecular weights of the polar (hydrophilic) and non-polar (hydrophobic) parts of the molecule is greater than 1.5, and (iii) a cationic polymer having a molecular weight of at least 100,000 Dalton and a cationic charge density of at least 0.5 meq/g; in which the weight ratio of (i):(ii) ranges from 3:1 to 1:3. Shampoo compositions according to the invention demonstrate reduced extensional viscosity (i.e. are less “stringy”). They also provide for the delivery of a significantly increased level of conditioning without any significant increase in product stringiness.

### FIELD OF THE INVENTION

This invention relates to shampoo compositions containing cationic polymer and an anionic surfactant mixture, and more particularly to shampoo compositions which incorporate an anionic surfactant mixture in which the anionic surfactants are differentiated by the ratio between the molecular weights of the polar (hydrophilic) and non-polar (hydrophobic) parts of the molecule, so that when combined with the cationic polymer, reduced levels of “stringiness” are observed.

### BACKGROUND AND PRIOR ART

Shampoo compositions comprising various combinations of cleansing surfactant and conditioning agents are known. These products typically comprise an anionic cleansing surfactant in combination with a conditioning agent. Amongst the most popular conditioning agents used in shampoo compositions are oily materials such as mineral oils, naturally occurring oils such as triglycerides and silicone polymers. These are generally present in the shampoo as dispersed hydrophobic emulsion droplets. Conditioning is achieved by the oily material being deposited onto the hair resulting in the formation of a film.

However, many shampoo compositions do not provide a sufficient level of deposition of conditioning agent onto the hair and skin during the cleansing process. Without such deposition, large proportions of conditioning agent are rinsed away during the cleansing process and therefore provide little or no conditioning benefit.

One known method for improving deposition of a conditioning agent involves the use of certain cationic deposition polymers. These polymers may be synthetic or natural polymers that have been modified with cationic substituents.

The effectiveness of such polymers as deposition aids is related to their molecular weight and cationic charge density. Cationic polymers having a combination of high molecular weight (e.g. 1M Dalton or more) and high cationic charge density (e.g. 1.6 meq/g or more) are particularly effective deposition aids. A problem is that such polymers also cause standard shampoo bases to develop very high extensional viscosity, meaning that the shampoo becomes very ‘stringy’. This is not only aesthetically undesirable, it can cause serious practical problems with filling bottles and especially sachets.

The present inventors have found that this problem can be solved if the cationic polymer is formulated with an anionic surfactant mixture in which the anionic surfactants are differentiated by the ratio between the molecular weights of the polar (or hydrophilic) and non-polar (hydrophobic) parts of the molecule.

Shampoo compositions according to the invention demonstrate reduced extensional viscosity (i.e. are less “stringy”), which leads to better aesthetic properties and improved ease of handling during processing, especially during bottle and sachet filling.

Shampoo compositions according to the invention also provide for the delivery of a significantly increased level of conditioning without any significant increase in product stringiness.

### SUMMARY OF THE INVENTION

The present invention provides an aqueous shampoo composition comprising, in admixture:
(i) a first anionic surfactant in which the ratio between the molecular weights of the polar (hydrophilic) and non-polar (hydrophobic) parts of the molecule is less than 1.5;
(ii) a second anionic surfactant in which the ratio between the molecular weights of the polar (hydrophilic) and non-polar (hydrophobic) parts of the molecule is greater than 1.5, and
(iii) a cationic polymer having a molecular weight of at least 100,000 Dalton and a cationic charge density of at least 0.5 meq/g;

in which the weight ratio of (i):(ii) ranges from 3:1 to 1:3.

### DETAILED DESCRIPTION

All molecular weights as used herein are weight average molecular weights, unless otherwise specified.

The term “cationic charge density”, as used herein, refers to the ratio of the number of positive charges on a monomeric unit of which a polymer is comprised to the molecular weight of the monomeric unit. The charge density multiplied by the polymer molecular weight determines the number of positively charged sites on a given polymer chain.

By “aqueous shampoo composition” is meant a composition which has water or an aqueous solution or a lyotropic liquid crystalline phase as its major component. Suitably, the composition will comprise from 50% to 98% by weight based on total weight of water, preferably from 60% to 90%.

First Anionic Surfactant

Shampoo compositions according to the invention comprise a first anionic surfactant in which the ratio between the molecular weights of the polar (hydrophilic) and non-polar (hydrophobic) parts of the molecule is less than 1.5. Examples of suitable first anionic surfactants are the alkyl sulphates and alkyl ether sulphates and salts thereof, especially their sodium, magnesium, ammonium and mono-, di- and triethanolamine salts. The alkyl groups generally contain from 8 to 18, preferably from 10 to 16 carbon atoms and may be unsaturated. The alkyl ether sulphates may contain from 1 to 20 ethylene oxide or propylene oxide units per molecule.

Preferred first anionic surfactants are sodium lauryl sulphate, sodium lauryl ether sulphate(n)EO, (where n is from 1 to 3), ammonium lauryl sulphate and ammonium lauryl ether sulphate(n)EO, (where n is from 1 to 3).

A particularly preferred first anionic surfactant is sodium lauryl ether sulphate(n)EO (where n is 1).

Mixtures of any of the foregoing first anionic surfactants may also be suitable.

The total amount of first anionic surfactant in shampoo compositions of the invention generally ranges from 1 to 25%, preferably from 4 to 12%, more preferably from 4 to 8% by weight based on the total weight of the composition.

Second Anionic Surfactant

Shampoo compositions according to the invention comprise a second anionic surfactant in which the ratio between the molecular weights of the polar (hydrophilic) and non-polar (hydrophobic) parts of the molecule is greater than 1.5.

Examples of suitable second anionic surfactants are sulfosuccinate anionic surfactants.

Preferred sulfosuccinate anionic surfactants have the general formula:
R—(O—CH₂—CH₂—)ₓ—O—CH(O)—CH(SO₃⁻M⁺)-CH₂—CH(O)—O-M′⁺
in which R is a straight or branched chain alkyl or alkenyl group having 10 to 22 carbon atoms, X is a number that represents the average degree of ethoxylation and can suitably range from 0 to 5, preferably from 0 to 4, most preferably from 2 to 3.5, and M and M′ are monovalent cations which can be the same or different from each other. Preferred cations are alkali metal ions such as sodium or potassium, ammonium ions, or alkanolammonium ions such as monoethanolammonium or triethanolammonium ions.

Most preferred sulfosuccinate surfactants are C₁₀-C₁₄ sulfosuccinate and C₁₀-C₁₄ ethoxy (1-5) sulfosuccinate, especially laureth-3 sulfosuccinate.

Mixtures of any of the foregoing second anionic surfactants may also be suitable.

The total amount of second anionic surfactant in shampoo compositions of the invention generally ranges from 1 to 20%, preferably from 4 to 12%, more preferably from 4 to 8% by weight based on the total weight of the composition.

The weight ratio of first anionic surfactant to second anionic surfactant in compositions of the invention ranges from 3:1 to 1:3. It preferably ranges from 2:1 to 1:2 and is most preferably about 1:1.

Cationic Polymer

Shampoo compositions according to the invention comprise a cationic polymer having a molecular weight of at least 100,000 Dalton and a cationic charge density of at least 0.5 meq/g.

By “cationic polymer” is meant any polymer containing cationic groups and/or groups that can be ionized into cationic groups.

Suitable cationic polymers may be homopolymers or may be formed from two or more types of monomers.

The weight average (M_{w}) molecular weight of the cationic polymer is preferably between 300,000 and 2M Dalton, more preferably between 750,000 and 1.5M Dalton.

The cationic groups will generally be present as a substituent on a fraction of the total monomers of the cationic polymer. Thus when the polymer is not a homopolymer it can contain non-cationic spacer monomers. Such polymers are described in the CTFA Cosmetic Ingredient Dictionary, 3rd edition. The ratio of the cationic to non-cationic monomers is selected to give polymers having a cationic charge density in the required range.

The cationic charge density of the cationic polymer may suitably be determined via the Kjeldahl method as described in the US Pharmacopoeia under chemical tests for nitrogen determination. Preferred cationic polymers will have cationic charge densities of at least about 0.9 meq/gm, more preferably at least about 1.6 meq/gm, most preferably at least about 1.8 meq/g, but also preferably less than about 7 meq/gm, more preferably less than about 5 meq/gm, most preferably less than about 3.0 meq/g, as measured at the pH of intended use of the shampoo composition. The pH of intended use of the shampoo composition typically ranges from about pH 3 to about pH9, preferably from about pH4 to about pH7.

Any anionic counterions may be use in association with the cationic polymers so long as the cationic polymers remain soluble in the shampoo composition, and so long as the counterions are physically and chemically compatible with the essential components of the shampoo composition or do not otherwise unduly impair product performance, stability or aesthetics. Examples of such counterions include: halides (e.g., chloride, fluoride, bromide, iodide), sulfate, methylsulfate, and mixtures thereof.

The preferred cationic polymers are chosen from those that contain units comprising primary, secondary, tertiary and/or quaternary amine groups that can either form part of the main polymer chain or can be borne by a side substituent directly connected thereto.

Suitable cationic polymers may be naturally-derived materials such as cationic polysaccharides.

Cationic polysaccharides suitable for use in compositions of the invention include monomers of the formula:
A-O—[R—N⁺(R¹)(R²)(R³)X⁻],
wherein: A is an anhydroglucose residual group, such as a starch or cellulose anhydroglucose residual. R is an alkylene, oxyalkylene, polyoxyalkylene, or hydroxyalkylene group, or combination thereof. R¹, R² and R³ independently represent alkyl, aryl, alkylaryl, arylalkyl, alkoxyalkyl, or alkoxyaryl groups, each group containing up to about 18 carbon atoms. The total number of carbon atoms for each cationic moiety (i.e., the sum of carbon atoms in R¹, R² and R³) is preferably about 20 or less, and X is an anionic counterion.

Preferred cationic polysaccharides are cationic cellulose derivatives such as those salts of hydroxyethyl cellulose reacted with trimethyl ammonium substituted epoxide, referred to in the industry (CTFA) as Polyquaternium 10. Specific examples of these materials include those polymers available from Amerchol Corporation in their Polymer JR series of polymers, such as Polymer JR125, Polymer JR400 and Polymer JR30M. Other suitable types of cationic cellulose include the polymeric quaternary ammonium salts of hydroxyethyl cellulose reacted with lauryl dimethyl ammonium-substituted epoxide referred to in the industry (CTFA) as Polyquaternium 24.

Another preferred class of cationic polysaccharide that can be used is a cationic guar gum derivative, especially guar hydroxypropyltrimethylammonium chloride. Specific examples of these materials include those polymers available from Rhodia in their JAGUAR series of polymers, such as JAGUAR C13S and JAGUAR C17.

Suitable cationic polymers may also be synthetically-derived materials such as those formed from vinyl monomers having cationic amine or quaternary ammonium functionalities, optionally together with non-cationic spacer monomers.

Suitable non-cationic spacer monomers include (meth)acrylamide, alkyl and dialkyl (meth)acrylamides, alkyl (meth)acrylate, vinyl caprolactone and vinyl pyrrolidine.

The alkyl and dialkyl substituted monomers preferably have C₁-C₇ alkyl groups, more preferably C₁₋₃ alkyl groups. Other suitable water soluble spacer monomers include vinyl esters, vinyl alcohol, maleic anhydride, propylene glycol and ethylene glycol.

Suitable vinyl monomers having cationic amine or quaternary ammonium functionalities include dialkylaminoalkyl acrylate, dialkylaminoalkyl methacrylate, dialkylaminoalkyl acrylamide, dialkylaminoalkyl methacrylamide, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts. The alkyl portions of these monomers are preferably lower alkyls such as the C1, C2 or C3 alkyls.

Examples of suitable cationic polymers formed from the above types of monomer include copolymers of 1-vinyl-2-pyrrolidone and 1-vinyl-3-methylimidazolium salt (e.g. chloride salt) (referred to in the industry by the Cosmetic, Toiletry, and Fragrance Association, “CTFA”, as Polyquaternium-16); copolymers of 1-vinyl-2-pyrrolidone and dimethylaminoethyl methacrylate (referred to in the industry by CTFA as Polyquaternium-11); cationic diallyl quaternary ammonium-containing polymers, including, for example, dimethyldiallylammonium chloride homopolymer, copolymers of acrylamide and dimethyldiallylammonium chloride (referred to in the industry by CTFA as Polyquaternium 6 and Polyquaternium 7, respectively); terpolymers of acrylic acid with dimethyldiallylammonium chloride and acrylamide (referred to in the industry by CTFA as Polyquaternium 39), and terpolymers of acrylic acid with methacrylamidopropyl trimethylammonium chloride and methyl acrylate (referred to in the industry by CTFA as Polyquaternium 47).

Preferred cationic polymers formed from the above types of monomer are those in which the vinyl monomers having cationic amine or quaternary ammonium functionalities conform to the formula: 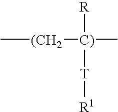
in which T is —O— or preferably —C(O)—, R is H or CH₃ and R¹ is:
—NH—(CH₂)ₙ—N⁺(R²)(R³)(R⁴)X⁻
in which n is an integer from 1 to 8, preferably 1 to 4, each of R², R³ and R⁴ are independently hydrogen or a short chain alkyl having from 1 to 4, preferably from 1 to 2 carbon atoms, and X is a counterion. The nitrogen attached to R², R³ and R⁴ may be a protonated amine (primary, secondary or tertiary), but is preferably a quaternary ammonium wherein each of R², R³ and R⁴ are alkyl groups.

Most preferred cationic polymers formed from the above types of monomer are those cationic polymers formed from methacrylamidopropyl trimonium chloride and/or acrylamidopropyl trimonium chloride and copolymers of these monomers with acrylamide. Specific examples of these materials include polymethyacrylamidopropyl trimonium chloride, available under the trade name Polycare 133, from Rhone-Poulenc, and acrylamidopyltrimonium chloride/acrylamide copolymer, available under the trade name SALCARE SC60 from Ciba Speciality Chemicals. The latter is a particularly effective deposition aid for conditioning agents.

Cationic polymer will generally be present in compositions of the invention at levels of from 0.01 to 5%, preferably from 0.05 to 1%, more preferably from 0.08 to 0.5% by total weight of cationic polymer based on the total weight of the composition.

Optional Ingredients

Optionally, the aqueous shampoo composition of the invention may contain further ingredients as described below to enhance performance and/or consumer acceptability.

Co-Surfactant

The composition can include co-surfactants, to help impart aesthetic, physical, cleansing or mildness properties to the composition.

A preferred example of a co-surfactant is an amphoteric surfactant, which can be included in an amount ranging from 0.5 to about 15%, preferably from 1 to 10%, most preferably from 1.5 to 5.5% by weight based on the total weight of the composition.

Examples of amphoteric surfactants include alkyl amine oxides, alkyl betaines, alkyl amidopropyl betaines, alkyl sulphobetaines (sultaines), alkyl glycinates, alkyl carboxyglycinates, alkyl amphoacetates, alkyl amphopropionates, alkylamphoglycinates, alkyl amidopropyl hydroxysultaines, acyl taurates and acyl glutamates, wherein the alkyl and acyl groups have from 8 to 19 carbon atoms. Typical amphoteric and zwitterionic surfactants for use in shampoos of the invention include lauryl amine oxide, cocodimethyl sulphopropyl betaine, lauryl betaine, cocamidopropyl betaine and sodium cocoamphoacetate.

A particularly preferred amphoteric surfactant is cocamidopropyl betaine.

Conditioning Agents

It is especially preferred that shampoo compositions of the invention comprise one or more conditioning agents to optimise wet and dry conditioning benefits.

Preferred conditioning agents are emulsified silicones.

Suitable emulsified silicones include those formed from silicones such as polydiorganosiloxanes, in particular polydimethylsiloxanes which have the CTFA designation dimethicone, polydimethyl siloxanes having hydroxyl end groups which have the CTFA designation dimethiconol, and amino-functional polydimethyl siloxanes which have the CTFA designation amodimethicone.

The emulsified silicone droplets may typically have a Sauter mean droplet diameter (D_{3, 2}) in the composition of the invention ranging from 0.01 to 20 micrometer, more preferably from 0.2 to 10 micrometer.

A suitable method for measuring the Sauter mean droplet diameter (D_{3, 2}) is by laser light scattering using an instrument such as a Malvern Mastersizer.

Suitable emulsified silicones for use in compositions of the invention are available as pre-formed silicone emulsions from suppliers of silicones such as Dow Corning and GE Silicones. The use of such pre-formed silicone emulsions is preferred for ease of processing and control of silicone particle size. Such pre-formed silicone emulsions will typically additionally comprise a suitable emulsifier, and may be prepared by a chemical emulsification process such as emulsion polymerisation, or by mechanical emulsification using a high shear mixer. Pre-formed silicone emulsions having a Sauter mean droplet diameter (D_{3, 2}) of less than 0.15 micrometers are generally termed microemulsions.

Examples of suitable pre-formed silicone emulsions include emulsions DC2-1766, DC2-1784, DC-1785, DC-1786, DC-1788 and microemulsions DC2-1865 and DC2-1870, all available from Dow Corning. These are all emulsions/microemulsions of dimethiconol. Also suitable are amodimethicone emulsions such as DC939 (from Dow Corning) and SME253 (from GE Silicones).

Also suitable are silicone emulsions in which certain types of surface active block copolymers of a high molecular weight have been blended with the silicone emulsion droplets, as described for example in WO03/094874. In such materials, the silicone emulsion droplets are preferably formed from polydiorganosiloxanes such as those described above. One preferred form of the surface active block copolymer is according to the following formula: 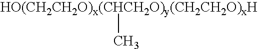
wherein the mean value of x is 4 or more and the mean value of y is 25 or more.

Another preferred form of the surface active block copolymer is according to the following formula: 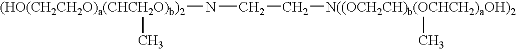
wherein the mean value of a is 2 or more and the mean value of b is 6 or more.

Mixtures of any of the above described silicone emulsions may also be used.

Silicone will generally be present in a composition of the invention at levels of from 0.05 to 10%, preferably 0.05 to 5%, more preferably from 0.5 to 2% by total weight of silicone based on the total weight of the composition.

Compositions according to the present invention may also comprise a dispersed, non-volatile, water-insoluble non-silicone oily conditioning agent.

The oily conditioning agent may suitably be selected from oily or fatty materials, and mixtures thereof.

Suitable oily or fatty materials are selected from hydrocarbon oils, fatty esters and mixtures thereof.

The oily or fatty material is suitably present in shampoo or conditioner compositions at a level of from 0.05 to 10, preferably from 0.2 to 5, more preferably from about 0.5 to 3 percent by weight of the composition.

Mixtures of any of the above described conditioning agents may also be used.

Suspending Agent

Preferably an aqueous shampoo composition of the invention comprises a suspending agent for optimising the physical stability of the shampoo, especially when the shampoo incorporates conditioning agents as described above such as emulsified silicones.

Suitable suspending agents are selected from polyacrylic acids, cross-linked polymers of acrylic acid, copolymers of acrylic acid with a hydrophobic monomer, copolymers of carboxylic acid-containing monomers and acrylic esters, cross-linked copolymers of acrylic acid and acrylate esters, heteropolysaccharide gums and crystalline long chain acyl derivatives. The long chain acyl derivative is desirably selected from ethylene glycol stearate, alkanolamides of fatty acids having from 16 to 22 carbon atoms and mixtures thereof. Ethylene glycol distearate and polyethylene glycol 3 distearate are preferred long chain acyl derivatives, since these impart pearlescence to the composition. Polyacrylic acid is available commercially as Carbopol 420, Carbopol 488 or Carbopol 493. Cross-linked polymers of acrylic acid are available commercially as Carbopol 910, Carbopol 934, Carbopol 941 and Carbopol 980. An example of a suitable copolymer of a carboxylic acid-containing monomer and acrylic acid esters is Carbopol 1342. All Carbopol (trademark) materials are available from Goodrich. Suitable cross-linked polymers of acrylic acid and acrylate esters are Pemulen TR1 or Pemulen TR2. A suitable heteropolysaccharide gum is xanthan gum, for example that available as Kelzan mu.

Mixtures of any of the above suspending agents may be used.

Preferred are cross-linked polymers of acrylic acid. Suspending agent will generally be present in a shampoo composition of the invention at levels of from 0.1 to 10%, preferably from 0.5 to 6%, more preferably from 0.9 to 4% by total weight of suspending agent based on the total weight of the composition.

Other Optional Ingredients

Aqueous shampoo compositions of the invention may contain other ingredients for enhancing performance and/or consumer acceptability. Such ingredients include fragrance, dyes and pigments, pH adjusting agents, pearlescers or opacifiers, viscosity modifiers, preservatives, and natural hair nutrients such as botanicals, fruit extracts, sugar derivatives and amino acids.

Mode of Use

The compositions of the invention are primarily intended for topical application to the hair and/or scalp of a human subject in rinse-off compositions, in order to provide cleansing while improving hair fibre surface properties such as smoothness, softness, manageability, cuticle integrity, and shine. The compositions provided by the invention are aqueous compositions, used by massaging them into the hair followed by rinsing with clean water prior to drying the hair. Optionally, a separate conditioning formulation may be applied after rinsing and before drying, but this may not be necessary as the compositions of the invention are intended to provide both cleansing and conditioning to the hair.

The invention is further illustrated with reference to the following, non-limiting examples, in which all percentages are by weight based on total weight unless otherwise specified.

### EXAMPLES

### Examples 1 and 2 and Comparative Examples A and B

A panel of 12 independent observers was recruited and each individual was invited to assess a series of four shampoos for ‘stringiness’. The shampoos denoted as Examples 1 and 2 respectively were shampoos according to the invention and the shampoos denoted as Comparative Examples A and B respectively were not according to the invention.

The panellists were asked to rank the shampoos (highest rank=most ‘stringy’, lowest rank=least ‘stringy’). The average rank for each product was calculated and these mean values were transformed to a ‘relative stringiness’ score by applying a linear transformation to the data. This transformation maps the data onto a scale between 0 and 1 where a score of 0 represents the lowest possible score (i.e. all the panellists rated the product to be the least ‘stringy’) and a score of 1 represents the highest possible score (i.e. all the panellists rated the product to be the most ‘stringy’).

Table 1 contains the relative stringiness scores for the four shampoos.

TABLE 1

| | | Standard Error |
|---|---|---|
| | | (a measure of |
| | Relative | uncertainty in the |
| | Stringiness | relative |
| Product | score | stringiness score) |
| Comparative | 0.45 | 0.09 |
| Example A⁽¹⁾ | | |
| Comparative | 0.97 | 0.036 |
| Example B⁽²⁾ | | |
| Example 1⁽³⁾ | 0.25 | 0.087 |
| Example 2⁽⁴⁾ | 0.6 | 0.062 |

⁽¹⁾Shampoo with 14 wt % sodium lauryl ether sulphate (2E0), 2 wt % cocamidopropyl betaine, 0.2 wt % JAGUAR C13S
⁽²⁾Shampoo with 14 wt % sodium lauryl ether sulphate (2E0), 2 wt % cocamidopropyl betaine, 0.2 wt % SALCARE SC6O
⁽³⁾Shampoo with 6 wt % sodium lauryl ether sulphate (1EO), 4 wt % disodium laureth sulfosuccinate, 3 wt % cocamidopropyl betaine, 0.2 wt % JAGUAR C13S
⁽⁴⁾Shampoo with 6 wt % sodium lauryl ether sulphate (1EO), 4 wt % disodium laureth sulfosuccinate, 3 wt % cocamidopropyl betaine, 0.2 wt % SALCARE SC6O

By comparing the relative stringiness scores of Comparative Example A vs. Example 1 and Comparative Example B vs. Example 2, it is clear that shampoos according to the invention are significantly less ‘stringy’ than the corresponding shampoos not according to the invention which contain the same cationic polymer.

### Examples 3 to 5

A series of three shampoos according to the invention were prepared incorporating emulsified silicone as a conditioning agent and different cationic polymers of varying charge density and molecular weight. The deposition of the silicone onto the hair from each shampoo was evaluated. The results are shown in Table 2.

TABLE 2

| | | Charge density | |
|---|---|---|---|
| | Mw of | of cationic | Deposition of |
| | cationic | polymer | silicone on |
| Product | polymer (kDa) | (meq/g) | hair (ppm) |
| Example | 300 | 0.8 | 600 |
| 3⁽⁵⁾ | | | |
| Example | 300 | 1.6 | 1350 |
| 4⁽⁶⁾ | | | |
| Example | >1000 | 1.9 | 2300 |
| 5⁽⁷⁾ | | | |

⁽⁵⁾Shampoo with 6 wt % sodium lauryl ether sulphate (1EO), 4 wt % disodium laureth sulfosuccinate, 3 wt % cocamidopropyl betaine, 2 wt % emulsified silicone, 0.2 wt % JAGUAR C13S
⁽⁶⁾Shampoo with 6 wt % sodium lauryl ether sulphate (1EO), 4 wt % disodium laureth sulfosuccinate, 3 wt % cocamidopropyl betaine, 2 wt % emulsified silicone, 0.2 wt % JAGUAR C17
⁽⁷⁾Shampoo with 6 wt % sodium lauryl ether sulphate (1EO), 4 wt % disodium laureth sulfosuccinate, 3 wt % cocamidopropyl betaine, 2 wt % emulsified silicone, 0.2 wt % SALCARE SC60

Table 2 shows that the use of high molecular weight, high charge density cationic polymers such as SALCARE SC60 is highly advantageous for silicone deposition and hence for the delivery of conditioning benefits.

However, as shown in Table 1, (Comparative Example B) shampoos not according to the invention containing SALCARE SC60 are extremely ‘stringy’. Shampoos containing SALCARE SC60 according to the invention (Example 2) are much less stringy than shampoos such as Comparative Example B.

Furthermore, the difference in stringiness between Example 2 (according to the invention with SALCARE SC60) and Comparative Example A (not according to the invention with JAGUAR C13S), is not significant.

It follows that the invention allows the delivery of a significantly increased level of conditioning without any significant increase in product stringiness.

1. An aqueous shampoo composition comprising, in admixture:
(i) a first anionic surfactant in which the ratio between the molecular weights of the polar (hydrophilic) and non-polar (hydrophobic) parts of the molecule is less than 1.5;
(ii) a second anionic surfactant in which the ratio between the molecular weights of the polar (hydrophilic) and non-polar (hydrophobic) parts of the molecule is greater than 1.5, and
(iii) a cationic polymer having a molecular weight of at least 100,000 Dalton and a cationic charge density of at least 0.5 meq/g;
in which the weight ratio of (i):(ii) ranges from 3:1 to 1:3.

2. A composition according to claim 1, in which the first anionic surfactant is selected from sodium lauryl sulphate, sodium lauryl ether sulphate(n)EO, (where n is from 1 to 3), ammonium lauryl sulphate and ammonium lauryl ether sulphate(n)EO, (where n is from 1 to 3).

3. A composition according to claim 2, in which the first anionic surfactant is sodium lauryl ether sulphate(n)EO (where n is 1).

4. A composition according to claim 1, in which the second anionic surfactant is a sulfosuccinate anionic surfactant having the general formula:
R—(O—CH₂—CH₂—)ₓ—O—CH(O)—CH(SO₃⁻M⁺)—CH₂—CH(O)—O^{-M′}⁺
in which R is a straight or branched chain alkyl or alkenyl group having 10 to 22 carbon atoms, X is a number that represents the average degree of ethoxylation and can suitably range from 0 to 5, preferably from 0 to 4, most preferably from 2 to 3.5, and M and M′ are monovalent cations which can be the same or different from each other.

5. A composition according to claim 4, in which the second anionic surfactant is a C₁₀-C₁₄ sulfosuccinate or a C₁₀-C₁₄ ethoxy (1-5) sulfosuccinate, preferably laureth-3 sulfosuccinate.

6. A composition according to claim 1, in which the weight ratio of first anionic surfactant to second anionic surfactant ranges from 2:1 to 1:2, and is preferably about 1:1.

7. A composition according to claim 1′ in which the weight average (M_{w}) molecular weight of the cationic polymer is between 300,000 and 2M Dalton, more preferably between 750,000 and 1.5M Dalton.

8. A composition according to claim 1, in which the cationic polymer has a cationic charge densities of at least about 0.9 meq/gm, more preferably at least about 1.6 meq/gm, most preferably at least about 1.8 meq/g.

9. A composition according to claim 8, in which the cationic polymer has a cationic charge density of less than about 7 meq/gm, more preferably less than about 5 meq/gm, most preferably less than about 3.0 meq/g.

10. A composition according to claim 1, in which the cationic polymer is a cationic polymer formed from methacrylamidopropyl trimonium chloride and/or acrylamidopropyl trimonium chloride.

11. A composition according to claim 10, in which the cationic polymer is a copolymer of methacrylamidopropyl trimonium chloride and/or acrylamidopropyl trimonium chloride with acrylamide.

12. A composition according to any claim 1, which comprises an amphoteric surfactant in an amount ranging from 0.5 to about 15%, preferably from 1 to 10%, most preferably from 1.5 to 5.5% by weight based on the total weight of the composition.

13. A composition according to claim 12, in which the amphoteric surfactant is selected from lauryl amine oxide, cocodimethyl sulphopropyl betaine, lauryl betaine, sodium cocoamphoacetate, and preferably cocamidopropyl betaine.

14. A composition according to claim 1, which comprises a conditioning agent selected from emulsified silicones, dispersed, non-volatile, water-insoluble non-silicone oily conditioning agents, and mixtures thereof.

15. A composition according to claim 14, which comprises emulsified silicone at a level of from 0.05 to 10%, preferably 0.05 to 5%, more preferably from 0.5 to 2% by total weight of silicone based on the total weight of the composition.
